(19) United States
(12) Patent Application Publication
(10) Pub. No.: US 2025/0302412 A1
(43) Pub. Date: Oct. 2, 2025

(54) LARGE VESSEL OCCLUSION DETECTION AND BRAIN TISSUE ASSESSMENT SYSTEM AND METHOD

(71) Applicant: Yince Loh, Seattle, WA (US)

(72) Inventor: Yince Loh, Seattle, WA (US)

(21) Appl. No.: 19/091,463

(22) Filed: Mar. 26, 2025

Related U.S. Application Data
(60) Provisional application No. 63/570,591, filed on Mar. 27, 2024.

(51) Int. Cl. A61B 6/50; A61B 6/00; A61B 6/03; A61B 6/46; G06T 7/00; G06T 7/11; G06V 20/52; G16H 10/60; G16H 30/40

(52) U.S. Cl. CPC A61B 6/504; A61B 6/032; A61B 6/461; A61B 6/501; A61B 6/507; A61B 6/5217; G06T 7/0014; G06T 7/11; G06V 20/52; G16H 10/60; G16H 30/40; G06T 2207/10081; G06T 2207/20076; G06T 2207/30016; G06T 2207/30104; G06V 2201/031

(57) ABSTRACT

A system and method to evaluate a patient's brain condition by looking at venous outflow. The system and method may be computerized and automated. The process of the method identifies a paired set of venous structures to analyze, and then selects and identifies a mirrored pair of regions of interest on the structures and calculates the Hounsfield units for each of these mirrored pair of regions of interest of the paired venous structures. The process then calculates a ratio of the Hounsfield units of the mirrored pair of regions of interest. The process uses the calculated ratio to provide the clinician information on the condition on the brain tissue of the patient and to assess whether a large vessel occlusion actually exists.

### PRIORITY DATA

This application claims priority from the following U.S. patent application: U.S. Provisional Application Ser. No. 63/570,591, filed on Mar. 27, 2024. The disclosure of such application is incorporated herein, by reference, in its entirety.

### BACKGROUND

Nearly 800,000 strokes occur in the US annually, and almost 3 million Americans are currently disabled from them. Stroke is the third leading cause of death in the US and is the leading cause of disability costing over $73 billion/year in the US alone. The most disabling and deadly ischemic strokes (i.e. lack of blood flow to the brain) result from large vessel occlusions (LVO's). Patients with LVO's have extremely poor outcomes without treatment and until recently, respond poorly to standard of care (tissue plasminogen activator, or tPA).

Over the years, studies have shown that endovascular therapy (EVT) is more effective than optimal medical management in treating LVO's. These studies have shown that earlier intervention produces better clinical outcomes. As a result, state health departments, National Accreditation Organizations, and system of care designers have developed designations for health care facilities so medical personnel know a health care facility's stroke treatment capabilities. These stroke capability designations distinguish between those providing EVT, which is often 24/7, versus those that provide standard non-EVT stroke care. This is important because only a portion of ischemic strokes result from LVO's, and EVT does not help the rest. Because it is important that a person suffering an ischemic stroke from an LVO be sent to the proper facility, it is important that they are assessed quickly and properly so they are sent to the proper facility to treat them. As part of this assessment, it would also be helpful to assess whether they are a viable candidate for a thrombectomy and to assess whether the brain tissue around the blockage is still viable brain.

Present analysis for detection of an LVO, before assessing brain tissue viability, is to use CT, specifically CT angiography (CTA). Obtaining a CT angiogram has become the standard of care for all suspected strokes. The current treatment paradigm is a time-consuming three step process: 1) identify patients with an LVO by CTA; 2) after identification of an LVO, send them to an EVT treatment facility, and 3) once at the EVT treatment facility, image the patient again, but this time image them using CT or MR perfusion (CTP or MRP) to assess the viability of the brain tissue. Step 1 has significant drawbacks: 1) there are often delays because it requires a radiologist to identify the LVO on the CTA; 2) there may be a false positive determination and 3) the present initial analysis of a CTA does not provide information to assess the viability of the brain tissue so a CTP or MRP has to be done later. Specifically, the assessment of the brain tissue does not happen until step 3, after a patient is transferred to an EVT treatment facility. Even then, at step 3, to do the additional testing inherently requires moving the patient from the stretcher to the CT gantry; back to the stretcher and then waiting for the CTP to be processed and interpreted.

Accordingly, there is a need for an LVO detection and brain tissue assessment system and method to expedite and improve LVO identification and provide information on brain tissue viability at the time of the initial assessment so a practitioner has the information he needs at the time of initial assessment, and subsequent reimaging, using CTP or MRP, is no longer needed when the patient arrives at the EVT treatment facility.

### SUMMARY

According to one aspect of the present invention, a method for automated assessment of brain tissue of a patient includes the steps of identifying a computed tomography angiography dataset for the patient; from the identified computed tomography angiography dataset, identifying a paired set of venous structures in the brain; within the identified paired set of venous structures, selecting a pair of mirrored homogenous regions of interest; for each of the selected mirrored regions of interest, calculating the Hounsfield units for each region of interest; determining a ratio of the Hounsfield units calculated for each mirrored region of interest; analyzing the determined ratio to make an assessment of the condition of the brain tissue of the patient; and generating an output that includes at least one of a displayed image of the analyzed brain tissue of the patient, illustrating the selected pair of mirrored regions of interest of the venous structures and the calculated Hounsfield units.

According to another aspect of the present invention, a large vessel occlusion detection and brain tissue assessment system for a patient includes a stored computed tomography angiography dataset; a processor; a large vessel occlusion detection and brain tissue assessment module, where the module is configured to interact with the stored computed tomography angiography dataset to identify a dataset for the patient; use the identified computed tomography angiography dataset to identify a paired set of venous structures in the brain; from within the identified paired set of venous structures, select a pair of mirrored homogenous regions of interest; for each of the selected mirrored regions of interest, calculate the Hounsfield units for each region of interest; determine a ratio of the Hounsfield units calculated for each mirrored region of interest; and analyze the determined ratio to make an assessment of the condition of the brain tissue of the patient.

According to yet another aspect of the present invention, a non-transitory computer readable storage medium comprising having stored thereon a computer program comprising instructions that, when executed by a computer, cause the computer to identify a computed tomography angiography dataset for the patient; use the identified computed tomography angiography dataset to identify a paired set of venous structures in the brain; from within the identified paired set of venous structures, select a pair of mirrored homogenous regions of interest; for each of the selected mirrored regions of interest, calculate the Hounsfield units for each region of interest; determine a ratio of the Hounsfield units calculated for each mirrored region of interest; and analyze the determined ratio to make an assessment of the condition of the brain tissue of the patient.

### DRAWINGS

Objects, features, and advantages of the present invention will become apparent upon reading the following description in conjunction with the drawing figures, in which:

FIG. 1 is a block diagram of an embodiment of a detection and assessment system of the present invention;

FIG. 2 depicts an embodiment of the steps of a process of the present invention;

FIG. 3 shows a CTA angiogram in a normal patient, with a line down the interhemispheric divide, with identified venous structures (internal cerebral vein);

FIG. 3A shows the left hemisphere only of FIG. 3, taken down the interhemispheric divide;

FIG. 3B shows the right hemisphere only of FIG. 3, taken down the interhemispheric divide;

FIG. 4 shows a CTA angiogram in a patient with an LVO and the identified venous structures (internal cerebral vein), 2A and 2B;

FIG. 5 shows a CTA angiogram in a patient with an LVO and the identified venous structures (middle cerebral vein), 3A and 3B; and

FIG. 6 shows a CTA angiogram in a patient with an LVO and the identified venous structures (basal vein of Rosenthal), 4A and 4B.

### DESCRIPTION

Present LVO CTA analysis focuses on analyzing the arteries and analyzing the CTA to find where the LVO is that is blocking the artery. This step is necessary, but by focusing solely on the arteries, there is no confirmation of the identification of the LVO blockage, sometimes resulting in false positives. Additionally, there is no information on the viability of the brain tissue, which does not allow the health care provider, at the time the time of the initial CTA, to make an assessment of whether the patient is a good candidate for EVT, or how long the patient's brain can tolerate the LVO before becoming irreversibly damaged by a stroke.

Recent studies have shown that attention to the venous status can also be important in assessing a suspected LVO patient. CTA assessment of collaterals inherently does not measure tissue perfusion or its ability to withstand progression to permanent stroke. As such, recent analysis has moved to uncouple venous outflow CTA analysis from arterial CTA analysis because it can provide additional information that a sole arterial CTA analysis cannot by itself, because venous outflow is as important to understanding brain physiology as the arteries.

While the arteries are the physical structures that are blocked during LVO, current AI algorithms still have errors identifying them due to variations in human anatomy, and can be under-specific, i.e. have false positives. Certain paired venous structures have less variability in anatomy and can be used to add further specificity in automated CTA processing algorithms.

Venous outflow is dependent on several cerebral arterial-derived factors such as cerebral blood flow and cerebral blood volume. It is equally a measure of the collaterals within a given brain as these other known and named measures. Furthermore, because venous outflow is a single measurement obtained at a single point in time during the CTA, it can be automated in its identification, measurement, and interhemispheric comparison without the need for more complicated and less available CT perfusion or multiphase scanning. Hence, automated venous identification, measurement, and comparison (AVIMC) of the present invention can provide identification of a LVO as well as provide tissue-level perfusion and LVO-tolerance estimates at the time of an initial CTA, which may preclude the need for performing a time-consuming CT perfusion on arrival at a thrombectomy center.

The description below describes a system and method to post-process the first imaged CTA, focusing on venous information to expedite and improve LVO identification as well as provide tissue viability and LVO tolerance in order to obviate the need for subsequent reimaging using CTP or MRP at a later stage.

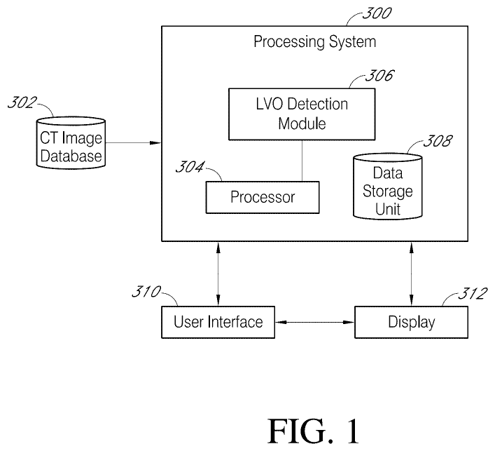

Referring to FIG. 1, according to an embodiment of the present invention, the system retrieves a captured CTA and processes the retrieved CTA using a venous identification and analytic process of an embodiment of the present invention. Referring specifically to FIG. 1, a block diagram of an embodiment of the detection and assessment system 300 of the present invention is depicted. The detection and assessment system 300 includes a processing system, which has, in addition to other known computing components, a processor 304, an LVO detection module 306 and a data storage unit 308. All of the components of the of the detection and assessment system 300 are in communication with each other and in communication with a CT image database 302, a user interface 310 and a display 312. It is understood that other known computing components and systems may be used with, or in communication with, the components of the embodiment of the invention described herein. The LVO detection module 306 may be configured to extract venous structure information from the CTA image data set and analyze such extracted venous structure information to detect an LVO and provide information to assess the present state of the patient's brain.

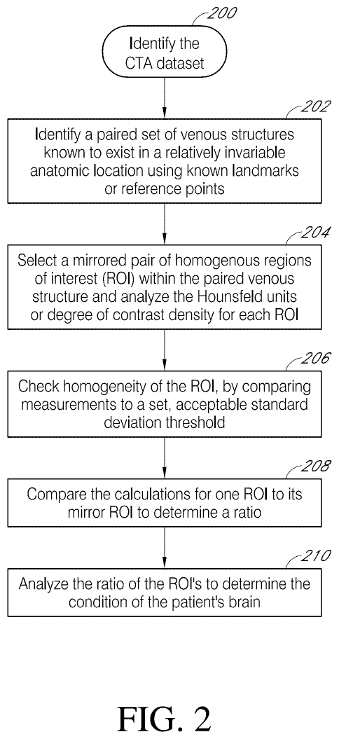

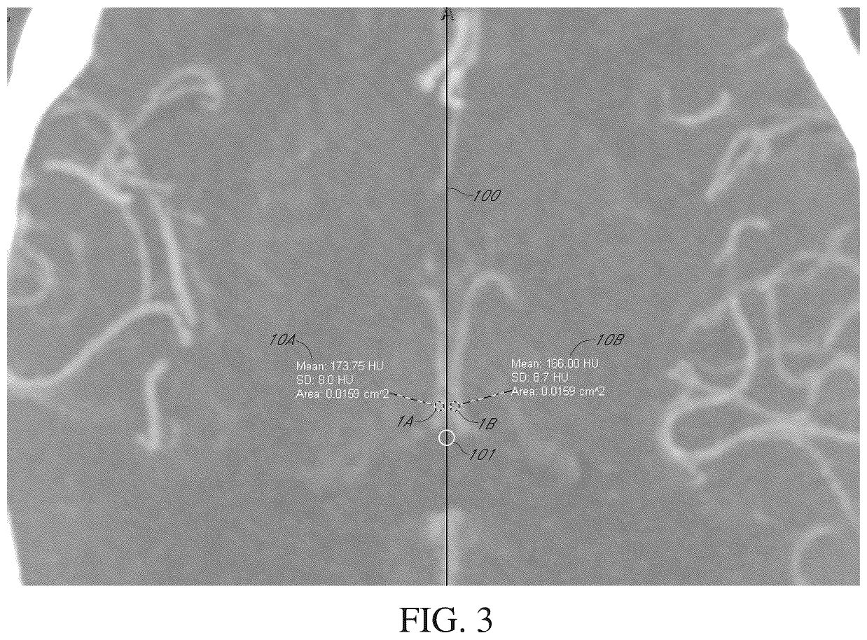

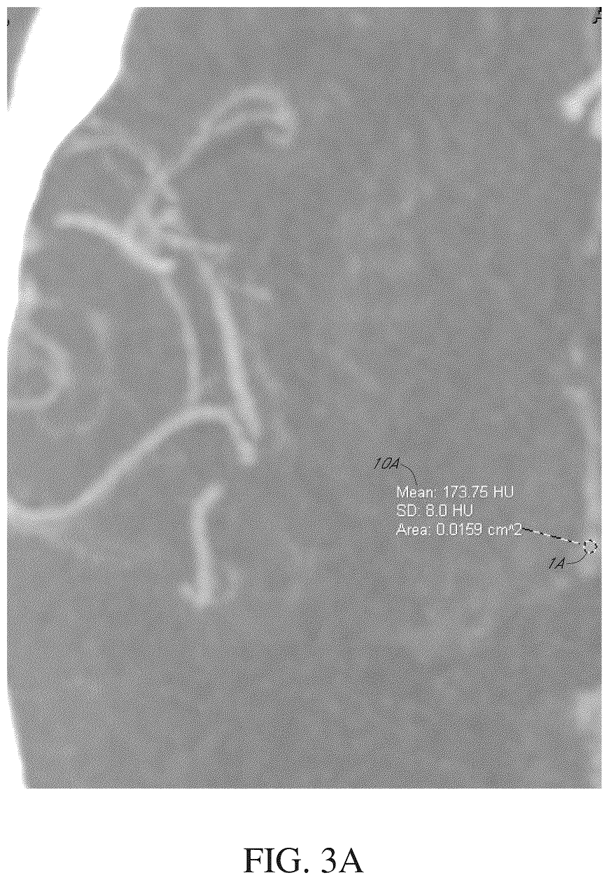

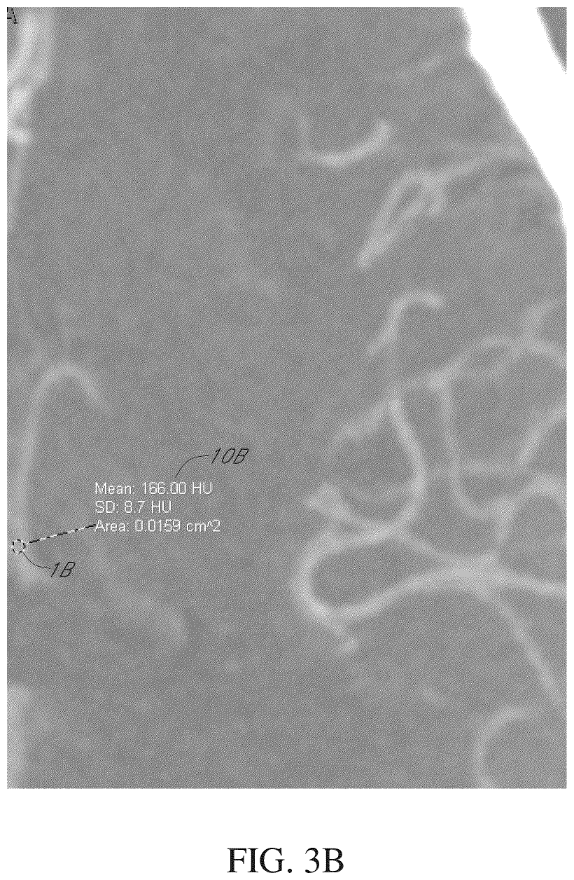

Referring now to FIG. 2, when the process of the LVO detection module 306 is activated, at step 200, the process identifies and retrieves a CTA data set for analysis from the CT image database 302. The CTA data sets of the CT image database 302, in this embodiment, have already been pre-processed for use with the process of the present invention. Then, at step 202, the process, using known landmarks or reference points, identifies a paired set of venous structures known to exist in a relatively invariable anatomic location. Referring to FIGS. 3, 3A and 3B, as an example, the process can identify the third ventricle as a linear midline structure 101 of low Hounsfield units (HU) (typically <15) that represents cerebrospinal fluid. In this example, with the third ventricle identified, the process can readily identify the paired internal cerebral veins (ICVs) from the analyzed axial CTA images as a pair of linear structures 1A, 1B running parallel to one another in the Anterior-Posterior (AP) plane 100 within the third ventricle 101. The middle cerebral veins (MCVs) and the basal veins of Rosenthal (BVRs) can be similarly identified using known invariable landmarks or reference points.

In this example, once the pair of venous structures 1A, 1B are identified, the process, at step 204, then selects a mirrored pair of homogenous regions of interest (ROI) 10A, 10B within the paired venous structure 1A, 1B and analyzes the Hounsfield units (HU) or degree of contrast density for each selected ROI. In one embodiment, the ROI is represented as a mean HU plus or minus a standard deviation (i.e. mean HU+/−SD). At step 206, the process checks homogeneity of the selected ROI's, by comparing measurements to a set, acceptable standard deviation threshold. Homogeneity is acceptable when the standard deviation is below a certain threshold. In one embodiment, the standard deviation is typically <10% to ensure that the ROI is drawn over only the vein and not including pixel measurements of adjacent non-venous structures. This is represented in the example depicted in FIGS. 3, 3A and 3B. As part of step 204, the process selects a first ROI 10A (FIG. 3A) and calculates the mean HU+/−SD. In this example, the process then identifies a mirror ROI 10B (FIG. 3B) of the first ROI 10A for the paired venous structure 1A, 1B. The process, to determine this mirror ROI 10B, utilizes the paired venous structure 1A, 1B and places the mirrored ROI 10B in a mirror plane down the AP axis 101; then identifying the closest ROI 10B. With the mirror ROI 10B identified, the process then calculates the mean HU+/−SD for the mirror ROI 10B.

Once a ROI 10A and its mirror ROI 10B are both selected, identified and measured at step 204, the homogeneity of the ROI's 10A, 10B are checked at step 206. Then, at step 208, the process compares the calculations for one ROI 10A to its mirror ROI 10B to determine a ratio. At step 210, the process then analyzes the determined ratio of the ROI's 10A, 10B, calculated at step 208, to determine the condition of the patient's brain, which can be displayed on the display 312, and presented in some other way, for use by the clinician to both confirm the existence of an LVO and to assess the brain tissue viability and tolerance to LVO of the patient's brain, so that a later CTP or MRP is not required.

By way of example, in the normal state, as depicted in FIGS. 3, 3A and 3B, the process determined that the ratio of ROI 10A to its mirror ROI 10B is approximately 1 (i.e. 166.00 HU/173.75 HU=0.96). In a deranged state, the ratio of ROI 10A to its mirror ROI 10B will not be 1. Also, it should be understood that this process of this invention is agnostic to which hemisphere or ROI contains the derangement, and hence the ratio, for the purposes of the embodiments described herein are always represented as less than one (i.e. <=1). In other words, the lower of the two ROI 10A, 10B values is always divided by the higher of the two ROI values. When the ratio is below a set threshold, at step 210, the process signals the detection of a deranged state. The degree of ratio lowering and hence derangement can represent the probability of the brain to tolerate the derangement over any pre-specified period of time. A lower ratio represents poor collaterals and a predicted rapid progression of stroke that will likely not benefit form EVT should the patient require transfer first.

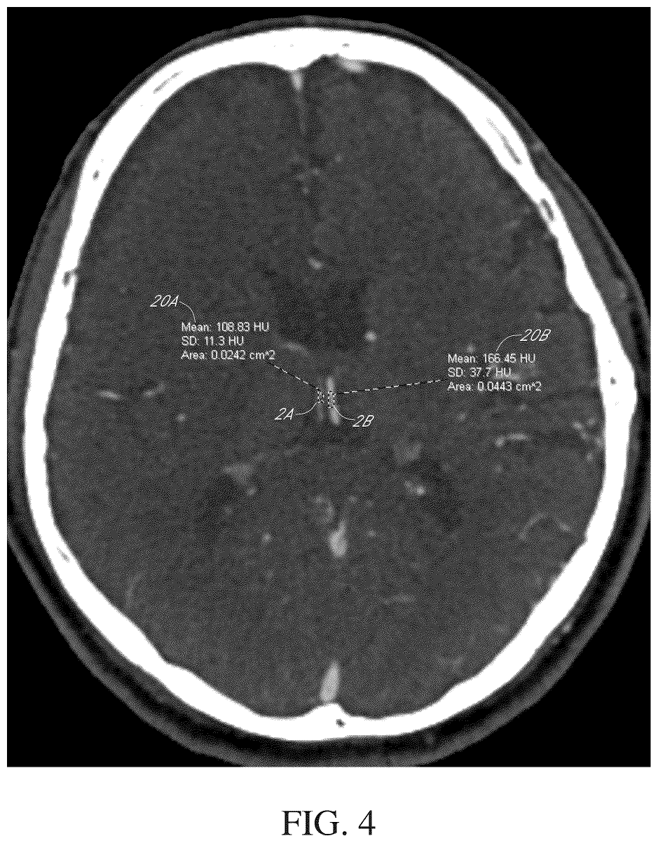

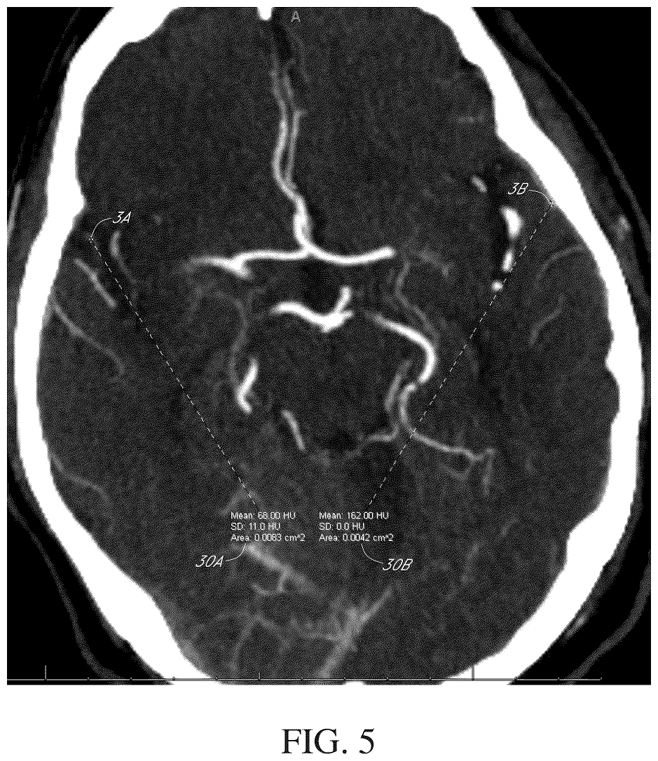

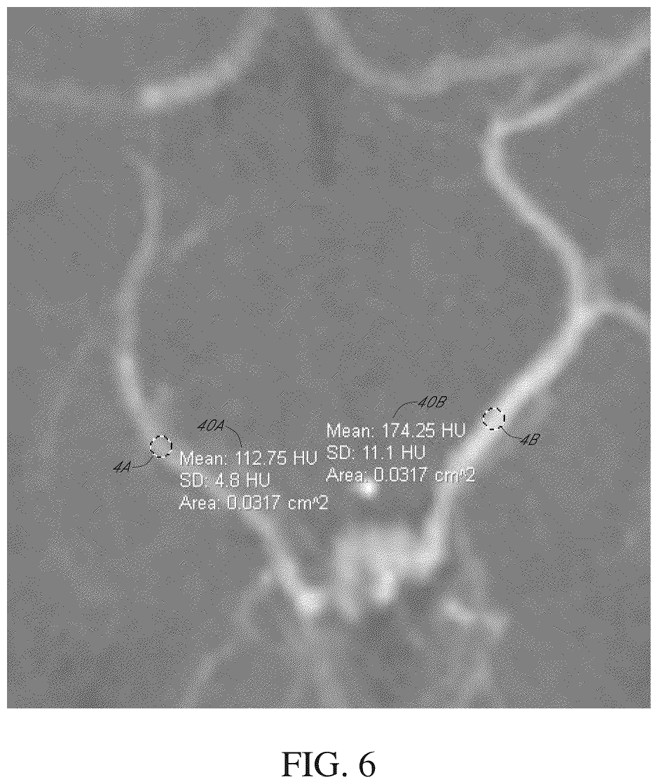

Further examples, described below, illustrate where the process of the present invention determined that the patient's brain is in a deranged state. FIG. 4 shows a CTA angiogram in a patient with an LVO and the identified venous structures (internal cerebral vein) 2A and 2B. The ROI's are selected and identified as 20A, 20B, and the process, at steps 204 and 206, calculates the mean HU+/−SD for each ROI 20A (Mean HU=108.83 HU), 20B (Mean HU=166.45 HU). At steps 208 and 210, the process calculates the interhemispheric ratio to be 0.65 (i.e. 20A divided by 20B; 108.83 HU/166.45 HU=0.65) and analyzes the condition of the brain and displays it to the user on the display 312. FIG. 5 shows a CTA angiogram in a patient with an LVO and the venous structures (middle cerebral vein) 3A and 3B identified by the process at step 202. The ROI's are selected and identified as 30A, 30B, and the process, at steps 204 and 206, calculates the mean HU+/−SD for each ROI 30A (Mean HU=68.00 HU), 30B (Mean HU=162.00 HU). At steps 208 and 210, the process calculates the interhemispheric ratio to be 0.42 (i.e. 30A divided by 30B; 68.00 HU/162.00 HU=0.42) and analyzes the condition of the brain and displays it to the user on the display 312. FIG. 6 shows a CTA in a patient with an LVO and the identified venous structures (basal vein of Rosenthal) 4A and 4B. The ROI's are selected and identified as 40A, 40B, and the process, at steps 204 and 206, calculates the mean HU+/−SD for each ROI 40A (Mean HU=112.75 HU), 20B (Mean HU=174.25 HU). At steps 208 and 210, the process calculates the interhemispheric ratio to be 0.65 (i.e. 40A divided by 40B; 112.75 HU/174.25 HU=0.65) and analyzes the condition of the brain and displays it to the user on the display 312.

In one embodiment, the process may first reconstruct the entire body of the CTA data into one three dimensional volumetric reconstruction, after which it may reinterpret axial, sagittal, or coronal slices once the data is “re-oriented” to exact X, Y, and Z coordinates using known landmarks such as the clinoid process, the temporal bone, or the orbits, as given examples. The system and method do not require “re-oriented” data to conduct venous analysis.

In another embodiment, the presence of a derangement itself below a pre-specified threshold can be used as an added calculation to improve the specificity and sensitivity of already commercially available software used to detect intracranial arterial large vessel occlusions, or “LVO detection”, software.

Although certain embodiments and features of an LVO detection and brain tissue assessment system and method have been described herein, the scope of coverage of this patent is not limited thereto. On the contrary, this patent covers all embodiments of the teachings of the disclosure that fairly fall within the scope of permissible equivalents.

What is claimed is:

1. A method for automated assessment of brain tissue of a patient, comprising the steps of:
identifying a computed tomography angiography dataset for the patient;
from the identified computed tomography angiography dataset, identifying a paired set of venous structures in the brain;
within the identified paired set of venous structures, selecting a pair of mirrored homogenous regions of interest;
for each of the selected mirrored regions of interest, calculating the Hounsfield units for each region of interest;
determining a ratio of the Hounsfield units calculated for each mirrored region of interest;
analyzing the determined ratio to make an assessment of the condition of the brain tissue of the patient; and
generating an output that includes at least one of a displayed image of the analyzed brain tissue of the patient, illustrating the selected pair of mirrored regions of interest of the venous structures and the calculated Hounsfield units.

2. The method of claim 1, wherein analyzing the determined ratio further includes confirming the existence of a large vessel occlusion.

3. The method of claim 1, wherein the identified paired set of venous structures in the brain are each internal cerebral veins.

4. The method of claim 1, wherein the identified paired set of venous structures in the brain are each middle cerebral veins.

5. The method of claim 1, wherein the identified paired set of venous structures in the brain are each basal veins of the Rosenthal.

6. The method of claim 1, further comprising the step of assessing the homogeneity of the selected paired regions of interest.

7. The method of claim 6, wherein calculating the Hounsfield units for each region of interest includes an allowable standard deviation.

8. The method of claim 7, wherein the allowable standard deviation is less than ten percent.

9. The method of claim 1, further comprising displaying the generated at least one of a displayed image of the analyzed brain tissue of the patient, illustrating the selected pair of mirrored regions of interest of the venous structures and the calculated Hounsfield units.

10. A large vessel occlusion detection and brain tissue assessment system for a patient, comprising:
a stored computed tomography angiography dataset;
a processor;
a large vessel occlusion detection and brain tissue assessment module, wherein the module is configured to:
interact with the stored computed tomography angiography dataset to identify a dataset for the patient;
use the identified computed tomography angiography dataset to identify a paired set of venous structures in the brain;
from within the identified paired set of venous structures, select a pair of mirrored homogenous regions of interest;
for each of the selected mirrored regions of interest, calculate the Hounsfield units for each region of interest;
determine a ratio of the Hounsfield units calculated for each mirrored region of interest; and
analyze the determined ratio to make an assessment of the condition of the brain tissue of the patient.

11. The system of claim 10, wherein the module is configured to analyze the determined ratio to further confirm the existence of a large vessel occlusion.

12. The system of claim 10, wherein the identified paired set of venous structures in the brain are each internal cerebral veins.

13. The system of claim 10, wherein the identified paired set of venous structures in the brain are each middle cerebral veins.

14. The system of claim 10, wherein the identified paired set of venous structures in the brain are each basal veins of the Rosenthal.

15. The system of claim 10, wherein the module is further configured to assess the homogeneity of the selected paired regions of interest.

16. A non-transitory computer readable storage medium comprising having stored thereon a computer program comprising instructions that, when executed by a computer, cause the computer to:
identify a computed tomography angiography dataset for the patient;
use the identified computed tomography angiography dataset to identify a paired set of venous structures in the brain;
from within the identified paired set of venous structures, select a pair of mirrored homogenous regions of interest;
for each of the selected mirrored regions of interest, calculate the Hounsfield units for each region of interest;
determine a ratio of the Hounsfield units calculated for each mirrored region of interest; and
analyze the determined ratio to make an assessment of the condition of the brain tissue of the patient.

17. The computer readable storage medium of claim 16, wherein the executed instructions analyze the determined ratio to further confirm the existence of a large vessel occlusion.

18. The computer readable storage medium of claim 16, wherein the identified paired set of venous structures in the brain are each internal cerebral veins.

19. The computer readable storage medium of claim 16, wherein the identified paired set of venous structures in the brain are each middle cerebral veins.

20. The computer readable storage medium of claim 16, wherein the identified paired set of venous structures in the brain are each basal veins of the Rosenthal.
